(19) United States
(12) Patent Application Publication
(10) Pub. No.: US 2015/0098906 A1
(43) Pub. Date: Apr. 9, 2015

(54) MEDICINALLY MARKED ROLLING PAPERS

(71) Applicant: Darrin B. Farrow, Westlake, OH (US)

(72) Inventor: Darrin B. Farrow, Westlake, OH (US)

(21) Appl. No.: 14/509,087

(22) Filed: Oct. 8, 2014

Related U.S. Application Data
(60) Provisional application No. 61/888,053, filed on Oct. 8, 2013.

(51) Int. Cl. A24D 1/02; A24C 5/10; A61K 9/00

(52) U.S. Cl. CPC A24D 1/02; A61K 9/0087; A24C 5/10
USPC 424/10.2; 131/280

(57) ABSTRACT

A method of manufacturing a cigarette and its constituent components including one or more formulations of filler materials and one or more rolling papers each printed with indicia indicative of the respective formulations of filler material. Also the provision of printed material that associates the particular indicia with its associated formulation.

### CROSS-REFERENCES TO RELATED APPLICATIONS

This application claims the benefit of U.S. Provisional application Ser. No. 61/888,053, filed on Oct. 8, 2013, and incorporated herein by reference.

### BACKGROUND

Rolling papers are used to prepare tobacco and medical marijuana into cigarettes for smoking for medical or recreational uses.

However, current rolling papers do not indicate the type of product or the formulation that is used in the cigarette, such as a daytime formula or nighttime formula, or the particular blend that was used, or amounts of active materials or drugs contained in the cigarette, potentially leading to confusion as to which cigarette has which formula, or the user must keep the cigarettes segregated from each other or manually mark such cigarettes. Such approaches are not ideal.

### SUMMARY

Provided are a plurality of example embodiments, including, but not limited to, rolling papers that are marked to indicate specific formulation of the filler material that is used to prepare the cigarette. Also provided is a method of manufacturing a cigarette using such marked rolling papers and associated filler material. Further provided is a method of manufacturing marked cigarette papers.

Also provided is method of manufacturing one or more cigarettes, comprising the steps of:
providing a first filler material with a first formulation which induces a first physical effect in a user;
providing a second filler material with a second formulation which induces a second physical effect in the user different than said first physical effect in type or intensity.
providing a first rolling paper printed with first indicia on the rolling paper indicative of the first formulation of filler material; and
providing a second rolling paper with second indicia on the rolling paper indicative of the second formulation of filler material.

Further provided is a method of manufacturing one or more cigarettes, comprising the steps of:
providing a first filler material with a first formulation which induces a first physical effect in a user;
providing a second filler material with a second formulation which induces a second physical effect in the user different than said first physical effect in type or intensity.
providing a first rolling paper printed with first indicia on the rolling paper indicative of the first formulation of filler material; and
providing a second rolling paper with second indicia on the rolling paper indicative of the second formulation of filler material;
providing first printed material associating the first filler material with the first indicial and describing or naming the first physical effect or an associated disease; and
providing second printed material associating the second filler material with the second indicia and describing or naming the second physical effect or an associated disease.

Also provided is a method of manufacturing one or more cigarettes, comprising the steps of:
preparing a first filler material with a first formulation which induces a first physical effect in a user;
preparing a second filler material with a second formulation which induces a second physical effect in the user different than said first physical effect in type or intensity.
printing a first indicia on a rolling paper to form a first rolling paper;
printing a second indicia on a rolling paper to form a second rolling paper;
rolling at least one first cigarette using the first filler material and the first rolling paper;
providing instructions associated with the first cigarette describing or naming the first physical effect or an associated disease;
rolling at least one second cigarette using the second filler material and the second rolling paper; and
providing instructions associated with the second cigarette describing or naming the second physical effect or an associated disease.

Further provided is a method of manufacturing a cigarette, comprising the steps of:
selecting a selected rolling paper having a particular indicia from among a plurality of different rolling papers each printed with different indicia representing a respective different formulation of filler material;
selecting a selected filler material from among a plurality of different filler materials each having a different formulation inducing a different physical effect in type or intensity, or for treating a different disease, wherein said particular indicial is indicative of the selected filler material; and
rolling at least one cigarette using the selected rolling paper and the selected filler material.

Also provided is a method of manufacturing one or more cigarettes, comprising the step of providing a rolling paper printed with first indicia on the first side the rolling paper indicative of a first formulation of filler material, said rolling paper also printed with a second indicia on the second side of the rolling paper indicative of a second formulation of filler material.

Further provided are additional example embodiments, some, but not all of which, are described hereinbelow in more detail.

### BRIEF DESCRIPTION OF THE DRAWINGS

The features and advantages of the example embodiments described herein will become apparent to those skilled in the art to which this disclosure relates upon reading the following description, with reference to the accompanying drawings, in which:

FIG. 1 is a schematic diagram of a first example marked rolling paper;

FIG. 2 is a schematic diagram of a second example marked rolling paper;

FIG. 3 is a schematic diagram of a third example marked rolling paper;

FIG. 4 is a schematic diagram of a fourth example marked rolling paper;

FIG. 5 is a schematic diagram of a fifth example marked rolling paper;

FIG. 6 is a schematic diagram of a sixth example marked rolling paper;

FIG. 7a is a schematic diagram of a seventh example marked rolling paper showing an amount of active ingredient;

FIG. 7b is a schematic diagram of an alternative of the embodiment of FIG. 7a showing another amount of active ingredient; and

FIGS. 8a, 8b, and 8c show schematic diagrams of example of cigarettes made using the example marked papers of FIGS. 7b, 2, and 6, respectively.

### DETAILED DESCRIPTION OF THE EXAMPLE EMBODIMENTS

Generally, a rolling paper is prepared in a manner similar to conventional manufacturing methods of manufacturing rolling papers by adding a step for printing indicia such as a word (using text) or an image (e.g., a symbol) on the rolling paper using an ink that is satisfactory and safe for use on a cigarette to be used by an individual or company to fill with a filling formulated (e.g., blended) for smoking for pleasure or for administering a dose of medication. The ink can be an ink that is approved for use on commercial cigarettes, for example, and should be an ink that has limited toxicity when burned. The ink may also be formulated to aid or at least no interfere with the burning process of the cigarette.

Alternatively, the paper may be printed with a watermark rather than using ink, in a process that is known in the art. Watermarks can also be adapted to aid in the burning process of the cigarette. As a further alternative, papers of different colors might be used for content indication, or papers colors might be combined with printed indicia for indicating more complex formulations or alternatives. Another alternative is to print using embossing or other types of stamping to provide the indicia by surface deformation.

The rolling paper manufacturing method should have the ability to print different indicia (e.g., images and/or text) on different batches of rolling papers so that these individual papers have different printing that can be used with respective different filling formulations to allow a user to easily distinguish the different filling formulations or types from each other. This additional manufacturing step may be combined with printing operations that are already being used for printing material on the papers, such as brand names, for example. However, it is important that there be at least two different types of printing to allow distinguishing the different formulations or types of filler.

Alternatively, the cigarette might have the distinguishing images or text printed on the cigarette after the cigarette has been manufactured by rolling the desired formulation in the papers prior to printing the identification material on the rolled cigarette. However, printing in advance is advantageous where a user may roll his own cigarettes for personal use, or where hand-rolling or limited batch machine production may be done in a customized retailer or drug dispensary. Cigarette rolling vendors providing automated rolling services for use by individuals have appeared in some areas to allow users to roll their own cigarettes, and hence marked papers would be useful for such a process.

Generally, the intention is to mark the papers in a manner that identifies the specific drug formulation, active ingredients (and/or if desired their amounts/potency), hybrid, blends, etc. to allow the user to identify the cigarette and differentiate it from different cigarettes which may have different formulations, blends, etc. For medical marijuana users, differently marked papers can be used for different strains of the plant for use to provide different medical effects.

For example, the Cannabis Sativa strain of marijuana is associated with more energetic and uplifting effects, and hence is a strain best adapted for daytime use. In contrast, the Cannabis Indica strain has mellowing, relaxing effects that can lead to sleepiness, and hence may be more appropriate for evening and nighttime use. At least one other strain of marijuana, Cannabis Ruderalis, also exists, but that tends to be less sought after for medical uses. Each of these strains may be indicated for different medical conditions, and hence may be desirable for use different times and doses.

Furthermore, different blends and hybrids of these strains can be developed to have still different effects. Marking the cigarette papers to identify the different strains, hybrids, blends, dopants, etc. either by using brand or strain names, or by marking to indicate their effects or purpose, can be particularly useful for medical marijuana users. For example, formulations of medical marijuana having controlled amounts of active substances (e.g., THC) or formulated for particular symptoms or diseases can us papers marked in a manner that identifies these distinctions.

Similarly, for tobacco users, one tobacco blend might be chosen having higher nicotine content for daytime use, and another blend might be chosen for bedtime use to avoid sleeplessness that might occur with high nicotine concentrations, since nicotine acts as a stimulant. Similarly, the nighttime blend might be used anytime the user is restless or nervous, and doesn't want to exacerbate those conditions. Furthermore, blends have successively less nicotine content could be used to wean the smoker from a cigarette addiction, for example, by choosing cigarette blends with less and less nicotine over time. Some blends may use dopants such as opioids to aid in sleep or pain relief, or other drugs for providing therapeutic effects or other desired physical or psychological or mental effects. Herbs may be added to have desired effects, flavors, or deodorizing effects, and flavorings could be similarly added.

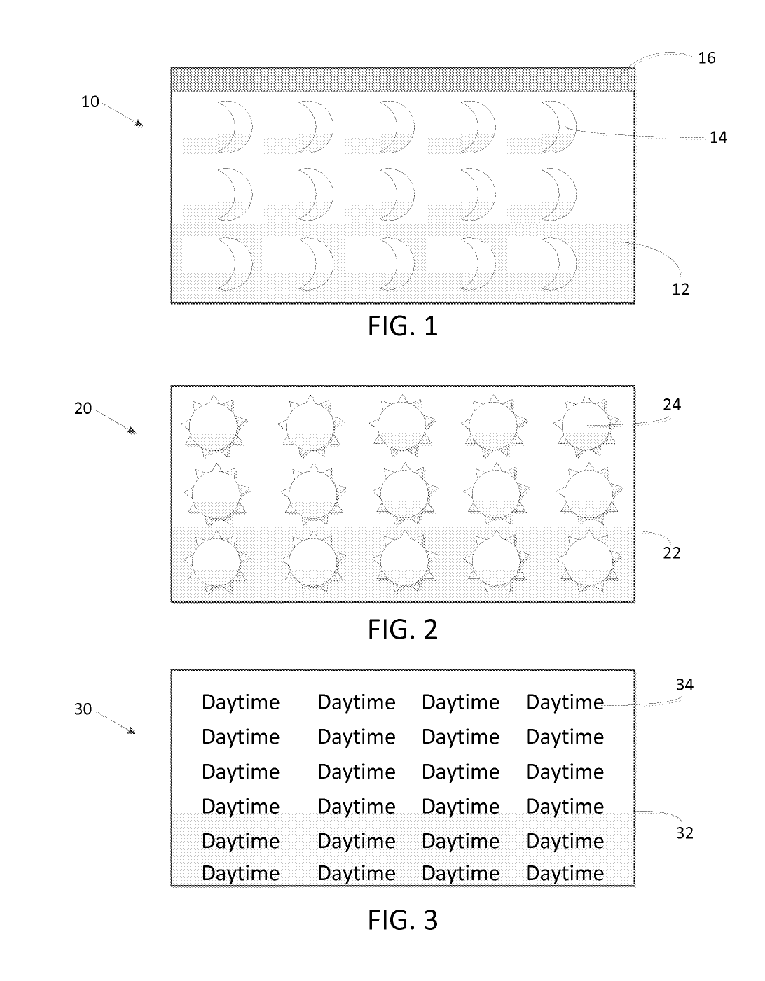

FIG. 1 is a drawing showing an example printed rolling paper 10 that has paper 12 that is marked with a plurality of graphical images 14 of a crescent moon, indicating that the resulting cigarette contains a filling that is formulated for use in the evening or at night. This example has a strip of glue 16 which is used to seal the cigarette sides and keep the cigarette together. Such a glue may be activated using saliva (i.e. it can be “licked”), or it may use some other type of glue. This glue may be provided on both sides of the paper (especially where the back side is printed with an alternative notification, as described below).

Nighttime formulations may include lower nicotine tobacco, or a filler that is doped with a sedative or sleep aid, for example, or a pain filler meant to last the entire night. For medical marijuana, an Indica blend may be utilized for its calming effect.

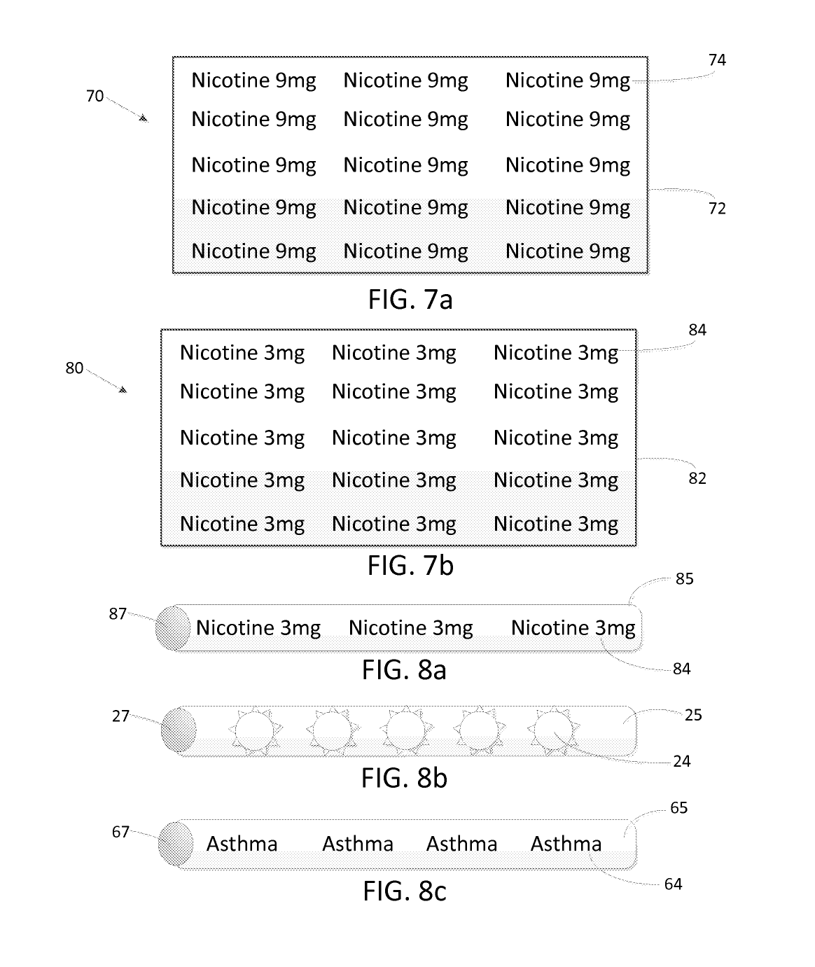

FIG. 2 is a drawing showing another example rolling paper 20 that has paper 22 marked with a plurality of images 24 of the sun, indicating that the resulting cigarette contains a filler that is formulated for use in the morning or during the day. Other types of graphical symbols could be used, such a marijuana leaf and a tobacco leaf to differentiate from the two different fillers, for example. FIG. 8b shows an example cigarette 25 with specially formulated filler 27 that was rolled using the printed paper of FIG. 7.

FIG. 3 shows an alternative to the example of FIG. 2, where instead of showing graphical suns, the printed rolling paper 30 is printed with the word “Daytime” 34 on the paper 32, to indicated that the resulting cigarette contains a filling that is formulated for use in the morning or during the day.

Note that although the rolling papers shown in FIGS. 1 and 2 may each be separate sets of papers having only one side printed, with the second side blank, an alternative approach would be to print the pattern shown in FIG. 1 on a first side of the paper, and the pattern shown in FIG. 2 on the second side of the paper, such that the proper side is shown on the outside of the cigarette to indicate the filling formula or blend that was used for rolling the cigarette.

This two-sided approach is particular useful when individuals will roll their own cigarettes, or where a self-rolling vendor is used, so that the individual need not purchase different rolling papers for rolling two different blends of fillings. For example, printing graphic images of a marijuana leaf on one side, and a tobacco leaf on the other side, would allow the user to use the same paper for rolling both marijuana cigarettes and tobacco cigarettes.

In this situation, glue used to seal the cigarette could be provided on both sides of the paper to allow the user to manually accomplish this task (e.g., by licking the seal).

The other printing embodiments discussed below could also be one sided or two sided, and any combination of two different printed patterns and/or text could be utilized as desired.

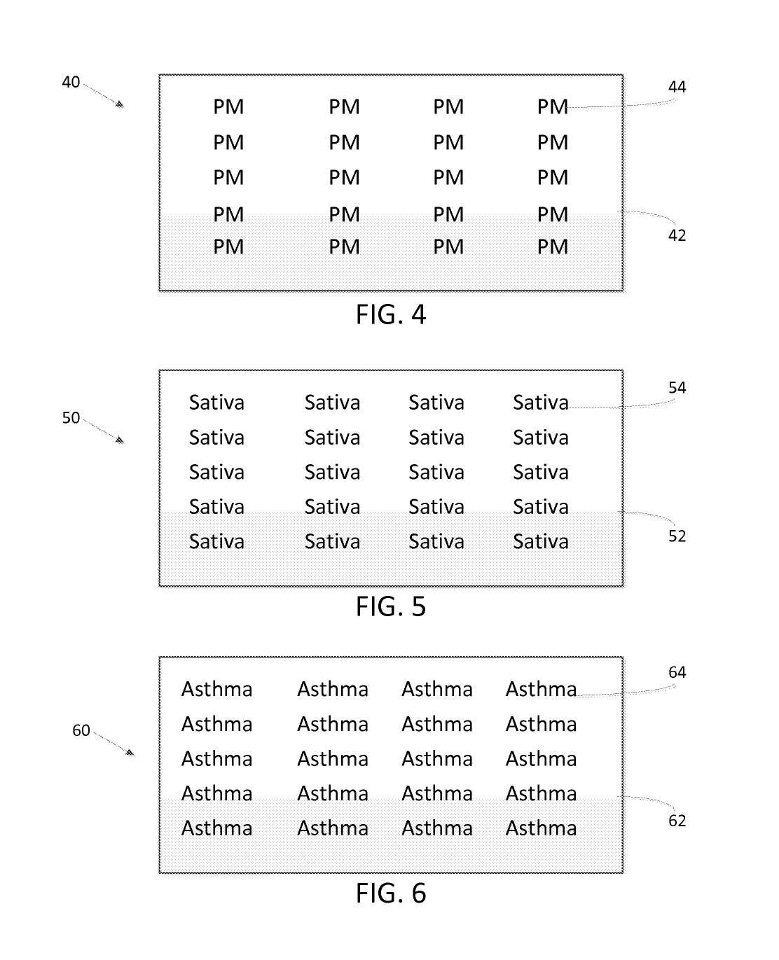

FIG. 4 is a drawing showing an example printed rolling paper 40 that has paper 52 printed with the abbreviation “PM” 54 to indicate that the resulting cigarette contains a filling that is formulated for the evening or at night. The other side of the paper might be printed with “AM” to indicate a morning or daytime formulation, for example.

FIG. 5 is a drawing showing an example printed rolling paper 50 that is printed with the word “Sativa” 54 on the paper 52, indicating that the resulting cigarette contains a filling that primarily contains the Sativa strain of marijuana. Furthermore, the other side could be printed with the word “Indica” for use in identifying that strain of marijuana.

FIG. 6 is a drawing showing an example printed rolling paper 60 that is printed with the word “Asthma” 64 on the paper 62, indicating that the resulting cigarette contains a filling that is formulated for those who suffer from asthma, such as spasmodic asthma. FIG. 8c shows an example cigarette 65 with specially formulated filler 67 rolled using the printed paper of FIG. 7.

FIG. 7a is a drawing showing an example printed rolling paper 70 that is printed with the phrase “Nicotine 9 mg” 74 on the paper 72, to indicate that the paper is used for a filler of common tobacco, which tends to have about 9 mg of nicotine per cigarette. This relatively high dosage of nicotine provides a stimulative effect on a user (smoker). Alternatively, the paper could be used to identify a cigarette that is specially formulated to administer that dosage of nicotine. FIG. 8a shows an example cigarette 85 with specially formulated filler 87 that was rolled using the printed paper of FIG. 7.

FIG. 7b is a drawing showing an example printed rolling paper 80 that is printed with the phrase “Nicotine 3 mg” 84 on the paper 82, to indicate that the paper is used for a filler of reduced nicotine tobacco. This relatively low dosage of nicotine avoids the stimulative effect of a higher dosage and hence can avoid sleeplessness or insomnia effects. Alternatively, the paper could be used to identify a cigarette that is specially formulated to administer that dosage of nicotine. This indicia could be printed on the opposite side of the paper of FIG. 7a, for example.

The specific nicotine amounts can be varied as desired, with higher amounts being provided for even more stimulative effect, or lower doses provided to have even less of a stimulative effect, with variations in-between supported as well. The nicotine can be varied by mixing various strains of tobacco, or by adding the drug to the tobacco or other filler as a dopant in precise measured quantities.

Furthermore, when such cigarettes are dispensed by a pharmacy, drug store, or doctor, the paper indicia may include other markings, such as the source of the fillers, warnings, FDA notices, or other information that may be desired, or either required by government regulation.

The rolling papers are shown in the example FIGs in a pre-cut form of a proper size for a single cigarette, which may be sold in booklets or packets, but the paper can also be provided in rolls of continuous length (many hundreds or thousands of feet) such as may be used by an automated manufacturing operation that cuts the paper during manufacture to make thousands of cigarettes very quickly.

The cigarette is made or manufactured by providing two or more different materials or formulations (e.g., different blends) to be used to fill the cigarette. If the papers are printed with the distinguishing markings in advance, then the cigarettes are manufactured by matching the proper formulation of filling material with the rolling papers that have the desired markings for indicating that formulation. For example, if a formulation is being used for aiding in sleep, the rolling papers that are marked with moons (e.g., FIG. 1) or “PM” indicators (e.g., FIG. 4) can be used, whereas daytime formulations may be marked with a sun (e.g., FIG. 2) or “Daytime” (e.g., FIG. 3). In contrast, if a particular strain of filling is being used, the papers might be marked with that strain (e.g., FIG. 5 referring to the “Sativa” strain). In this way, the user can differentiate different formulations of filler. Such manufacturing of the cigarettes may be done manually by an individual or on an assembly line, or it may be automated through the use of machines.

Alternatively, the cigarettes may be rolled with the desired filling materials, and then appropriately marked after being manufactured.

A printed “key” may be provided to map the images or printed material to the various formulations where there are a large number of such formulations, or where the meaning of the markings may not be obvious to the user.

For example, there may be a number of different cigarettes each providing different doses of medication for pain therapy. In such a case, the cigarettes might be labeled “PAIN” and numbered from 1 to 5, or 1 to 10, for example, with each consecutive number intended to be used for treating more intense pain. Or a formulation for treating a heart condition might have cigarettes marked with images of hearts, whereas a formulation for treating memory problems may have cigarettes marked with images of brains. Or the medications being administered by the formulations might be printed on the papers, such as “THC” for administering a filler utilizing pure THC (an active ingredient of marijuana), or other cannabinoids, for example.

As another example, a user may desire to use one formulation of a cigarette in the morning, and a different formulation before bed. This can be accomplished by using two differently marked cigarettes each having different formulations, one marked for daytime usage, and one marked for nighttime usage, avoiding any confusion on the part of the user as to which is which.

As a further alternative, the cigarette may be marked along its length with lines or numbers, (e.g., a linear measurement or scale) so that the user can control the administration of the active ingredients by controlling how much of the cigarette is smoked.

Any of the example rolling papers (or other covered embodiments) can be used in a number of ways for preparing cigarettes that can be easily identified as to contents by a user.

For example, a user or manufacturer may select a particular formulation of a filler material for rolling into cigarettes. This formulation may be provided by a pharmacy, distributor, retailer, farmer, or custom formulated by the user/manufacturer, for example. This formulation may include a blend of materials (e.g., tobacco, marijuana, hybrids thereof, or other base material or combinations thereof), and it may include the addition of controlled amount of drugs or other dopants (e.g., hash oil, THC or other cannabinoids; nicotine; sleep or pain agents such as opioids or opium, lung protective agents, asthma medications, etc.).

The formulation could be a blend of tobaccos with different properties, a blend of marijuana, such as a combination of Sativa and Indica strains, with or without hash oil or added cannabinoids. A Tobacco formulation may include added nicotine, or tobacco with all or part of the nicotine removed. A marijuana blend may include an opioid for pain, for example. Any of the blends may include other medications, such as for treating asthma (such as drugs used in asthma inhalers such as steroids or other drugs) or other lung problems, for example. Dopants might even include drugs for fighting cancers, such as a mouth, throat, tongue, or lung cancer. Other lung diseases such as emphysema, bronchitis, Cystic fibrosis, pneumonia, or COPD, for example might also be treated with specific drug and filler formulations, especially in smokers who refuse the give up the habit. Drugs or other materials to reduce the damage caused by smoking might also be treated using certain formulations. OCD, schizophrenia, depression, other psychological illnesses might also be treated by doping a tobacco or marijuana or other herbal filler with appropriate drugs for treating those disorders.

Some of these formulations might require production by a pharmacist or a licensed operator, whereas others may be prepared by an individual or an unlicensed entity. The formulations could come pre-packaged, or could be blended at the location of manufacture.

The paper is manufactured in a manner known in the art, but adapted to show an indicia that represents a formulation of filler for which the paper is to be used. This might be done by ink printing, water marking, embossing, or some other method of providing the desired indicia on the paper. The paper might be colored, as an alternative or additional feature.

Printed material may be provided to describe the specific formulations, their physical effects on a human, the disease(s) that they are intended to treat, their intensity, or other descriptions and/or instructions. This printed material can be provided with the formulated filler materials, or alternatively it may be provided with the rolling papers printed with the associated indicia. In some cases, the printed rolling papers, the filler formulations, and the printed materials may all be provided by a retail vendor, such as a marijuana dispensary or pharmacist, for example.

The user (an individual or manufacturer or other cigarette producer) can select an appropriate paper to identify the filler formulation that is to be used in the cigarette, or the user can order a customized indicia to be printed on rolling papers. The indicia on the paper might be text, a universally known symbol (e.g., sun or moon), a trademark, a medical symbol or text, a symbol requiring use of a key, a color, or some other indicia which may, or may not, intuitively identify the formulation or its intended use.

The user rolls the cigarette manually (using cut papers), or using an automated machine (using rolls of papers having continuous lengths) in the usual manner of rolling cigarettes, whether one-at-a-time, or in mass production. For situation where the paper is two-sided, the user will select the side appropriate for the intended formulation.

For example, a user might roll marijuana cigarettes containing the Indica strain of marijuana in a paper with “PM” or “Indica” or a moon printed on the outer side of the paper so that it is visible on the resulting rolled cigarette. The inner side might, in contrast, have printed on it “AM”, or “Sativa” or a sun, respectively. Conversely, the user might roll marijuana cigarettes containing the Sativa strain of marijuana in a paper with “AM” or “Sativa” or the sun printed on the outer side of the paper (and in the case of a double-sided printing, the inside might disclose “PM” or “Indica” or a moon, respectively).

As an alternatively, the cigarette might use unprinted papers, with the paper being marked after the cigarette is rolled. This might be done by printing, stamping, or embossing the indicia on the cigarette.

The resulting cigarettes should then show indicia on the outside of the cigarette that identifies the formulation that the cigarette is filled with. The cigarettes might be packaged in a pack or other container also marked with similar indicia to identify the formulations. These cigarettes might be used by the individual who rolled them, or distributed to retailers (such as drug stores or pharmacies) by a distributor, or sold by the manufacturer, such as a marijuana dispensary. The distribution and/or marketing may be subject to laws that may vary from state-to-state, or country-to-country, for example.

Many other example embodiments can be provided through various combinations of the above described features. Although the embodiments described hereinabove use specific examples and alternatives, it will be understood by those skilled in the art that various additional alternatives may be used and equivalents may be substituted for elements and/or steps described herein, without necessarily deviating from the intended scope of the application. Modifications may be necessary to adapt the embodiments to a particular situation or to particular needs without departing from the intended scope of the application. It is intended that the application not be limited to the particular example implementations and example embodiments described herein, but that the claims be given their broadest reasonable interpretation to cover all novel and non-obvious embodiments, literal or equivalent, disclosed or not, covered thereby.

What is claimed is:

1. A method of manufacturing one or more cigarettes, comprising the steps of:
providing a first filler material with a first formulation which induces a first physical effect in a user;
providing a second filler material with a second formulation which induces a second physical effect in the user different than said first physical effect in type or intensity.
providing a first rolling paper printed with first indicia on the rolling paper indicative of the first formulation of filler material; and
providing a second rolling paper with second indicia on the rolling paper indicative of the second formulation of filler material.

2. The method of claim 1, further comprising the step of rolling one or more cigarettes using the first filler material and the first rolling paper.

3. The method of claim 1, wherein said first formulation is a daytime formulation that avoids drowsiness or provides a stimulative effect.

4. The method of claim 3, wherein said first formulation includes a Sativa blend of marijuana.

5. The method of claim 3, wherein said first formulation includes a high stimulating dosage of nicotine.

6. The method of claim 1, wherein said second formulation is a nighttime formulation that induces drowsiness or provides a relaxing effect.

7. The method of claim 6, wherein said second formulation includes an Indica blend of marijuana.

8. The method of claim 1, wherein said first formulation includes a low stimulating dosage of nicotine.

9. The method of claim 1, wherein at least one of said formulations includes a drug for treating a disease.

10. The method of claim 1, wherein the step of providing the second rolling paper is provided by providing the first rolling paper with the first indicia printed on one side and the second indicia printed on the second side.

11. The method of claim 1, wherein at least one of said formulations includes a drug for treating a disease.

12. The method of claim 1, further comprising the step of providing printed material associated with the first filler material describing or naming the first physical effect or an associated disease.

13. The method of claim 1, wherein said first indicia represents a dosage or amount of an active ingredient in the first formulation.

14. A method of manufacturing one or more cigarettes, comprising the steps of:
providing a first filler material with a first formulation which induces a first physical effect in a user;
providing a second filler material with a second formulation which induces a second physical effect in the user different than said first physical effect in type or intensity.
providing a first rolling paper printed with first indicia on the rolling paper indicative of the first formulation of filler material; and
providing a second rolling paper with second indicia on the rolling paper indicative of the second formulation of filler material;
providing first printed material associating the first filler material with the first indicial and describing or naming the first physical effect or an associated disease; and
providing second printed material associating the second filler material with the second indicia and describing or naming the second physical effect or an associated disease.

15. The method of claim 14, further comprising the step of packaging all of said first filler material, said first rolling paper, and said first printed material for sale to a consumer.

16. The method of claim 14, further comprising the step of rolling one or more cigarettes using the first filler material and the first rolling paper.

17. A method of manufacturing one or more cigarettes, comprising the step of providing a rolling paper printed with first indicia on the first side the rolling paper indicative of a first formulation of filler material, said rolling paper also printed with a second indicia on the second side of the rolling paper indicative of a second formulation of filler material.

18. The method of claim 17, further comprising the steps of:
providing a first filler material with a first formulation which induces a first physical effect in a user; and
providing a second filler material with a second formulation which induces a second physical effect in the user different than said first physical effect in type or intensity.

19. The method of claim 18, further comprising the steps of: providing printed material associating the first filler material with the first indicial and describing or naming the first physical effect or an associated disease, and also associating the second filler material with the second indicia and describing or naming the second physical effect or an associated disease.

20. The method of claim 19, further comprising the step of packaging all of said first filler material, said second filler material, said rolling paper, and said printed material for sale to a consumer.

21. A method of manufacturing one or more cigarettes, comprising the steps of:
preparing a first filler material with a first formulation which induces a first physical effect in a user;
preparing a second filler material with a second formulation which induces a second physical effect in the user different than said first physical effect in type or intensity.
printing a first indicia on a rolling paper to form a first rolling paper;
printing a second indicia on a rolling paper to form a second rolling paper;
rolling at least one first cigarette using the first filler material and the first rolling paper;
providing instructions associated with the first cigarette describing or naming the first physical effect or an associated disease;
rolling at least one second cigarette using the second filler material and the second rolling paper; and
providing instructions associated with the second cigarette describing or naming the second physical effect or an associated disease.

22. A method of manufacturing a cigarette, comprising the steps of:
selecting a selected rolling paper having a particular indicia from among a plurality of different rolling papers each printed with different indicia representing a respective different formulation of filler material;
selecting a selected filler material from among a plurality of different filler materials each having a different formulation inducing a different physical effect in type or intensity, or for treating a different disease, wherein said particular indicial is indicative of the selected filler material; and
rolling at least one cigarette using the selected rolling paper and the selected filler material.
